(19) United States
(12) Patent Application Publication
(10) Pub. No.: US 2013/0144527 A1
(43) Pub. Date: Jun. 6, 2013

(54) DEVICE, SYSTEM AND METHOD FOR PERSONAL HEALTH MONITORING BASED ON MULTITUDE-POINTS ENVIRONMENTAL DATA

(71) Applicant: Irad Kuhnreichi, Haifa (IL)

(72) Inventor: Irad Kuhnreichi, Haifa (IL)

(21) Appl. No.: 13/817,002

(22) PCT Filed: Aug. 15, 2011
(86) PCT No.: PCT/IL11/00664

§ 371 Date: Feb. 14, 2013

Related U.S. Application Data
(60) Provisional application No. 61/373,832, filed on Aug. 15, 2010.

(51) Int. Cl. G06F 17/00; G01N 33/00

(52) U.S. Cl. CPC G06F 17/00; G01N 33/00
USPC 702/2

(57) ABSTRACT

A device, system and method for personal health monitoring based on multitude-points environmental data is disclosed. a plurality of indoor sensing devices and a plurality of outdoor sensing devices may be connected to a network to allow bidirectional communication between the sensing devices and a central server, and between the sensing devices themselves, over a communication network such as the internet. According to one method of the disclosed invention, a source of pollutions may be detected by comparing the levels of pollution sensed by different neighboring sensing devices and recommendations as to how to reduce the exposure to sensed pollutants may be sent to end users.

### BACKGROUND OF THE INVENTION

Pollution is the introduction of contaminants into an environment that causes instability, disorder, harm or discomfort to the ecosystem i.e. physical systems or living organisms. Pollution can take the form of chemical substances or energy, such as noise, heat, or light. Pollutants, the basic elements of pollution, can be foreign substances or energies, or naturally occurring. When naturally occurring, they are considered contaminants when they exceed natural levels. Pollution is often classed as point source or nonpoint source pollution.

In the late industrial age, the term over pollution was common, representing a view that was both critical of industrial pollution, but likewise accepted a certain degree of pollution as nominal industrial practice.

Air pollution is the introduction into the atmosphere, outdoors or indoors, of: chemicals, particulate matter, or biological materials that cause harm or discomfort to humans or to other living organisms, or damage the natural environment.

Air pollution, both indoors and outdoors, is a major environmental health problem affecting everyone in developed and developing countries alike. In order to cope with the health impacts of air pollution air quality guidelines are designed by the world health organization (WHO) and the environmental protection agency (EPA, USA). In regard to outdoor air pollution the WHO regulates four selected key elements: ground level Ozone (O3), Nitrogen dioxide (NO2), Sulfur Dioxide (SO2) and Particulate Matter (PM). The EPA regulates additional two elements: Lead (Pb) and Carbon monoxide (CO). The WHO Key findings in 2005 Air Quality Guidelines (applicable across all WHO regions):

(a) There are serious risks to health from exposure to Particulate Matter (PM) and Ozone (O3) in many cities of developed and developing countries. It is possible to derive a quantitative relationship between the pollution levels and specific health outcomes (increased mortality or morbidity). This allows invaluable insights into the health improvements that could be expected if air pollution is reduced.

(b) Even relatively low concentrations of air pollutants have been related to a range of adverse health effects.

(c) Poor indoor air quality may pose a risk to the health of over half of the world's population.

(d) Significant reduction of exposure to air pollution can be achieved through lowering the concentrations of several of the most common air pollutants emitted during the combustion of fossil fuels. Such measures will also reduce greenhouse gases and contribute to the mitigation of global warming.

Indoor air quality is a term referring to the air quality within and around buildings and structures, especially as it relates to the health and comfort of building occupants. Indoor air is becoming an increasingly more concerning health hazard than outdoor air. Indoor air pollution sources defer significantly between developed and developing countries. In developing countries the major source for pollutions is from heating and cooking based on biomass fuels together with improper ventilation or equipment. In developed countries sources that originate from modern consumer products and lifestyle are more relevant. The additional major elements of indoor air pollution, on top of those specified for outdoor pollution, are: Radon, molds, allergens, volatile organic compounds (VOC), asbestos fibers, carbon dioxide and formaldehyde. Indoor air pollution can be reduced by the use of active measures of various types (ventilation, air purifiers, selection of building materials, filters, plants etc.). The selection of proper means to reduce the indoor air pollution would better rely on indoor air pollution data readings that can accurately reflect the nature of the pollution.

The characteristic of outdoor air pollution is its variation in time and location. This time dependency originates from many factors and it includes both variation in hourly scale and in scale of longer period variation such as seasonal variations. Due to the high pollution emissions of modern transportation (road vehicles, airplanes, trains etc.) high pollution levels are subject to high transportation activity or to the geographical vicinity to it. From a personal point of view the meaning is: high exposure levels at locations of and during high transportation activity (for example; rush-hour, air port, picking from school etc.), where local high concentration of pollutants is likely. These events are characterized by short term, high pollution levels commonly called pollution peaks.

There are variety of active measures people can take to reduce pollution exposure and its risks. Examples are elevating indoor air quality by means of ventilation or air filtering. Air filtering could be done by mechanical means or by the use of plants and specialized building materials. Another sort of active measure that may be taken includes increasing dozes of medications to anticipated symptoms (mostly for unhealthy people).

### SUMMARY OF THE INVENTION

One of the system objectives is to reduce the negative health impacts of pollution on human health. The reduction of the negative impacts of air pollution may be achieved by giving users useful information and practical solutions relevant to their actual pollution exposure. Useful stands here for easy to understand to non professional people; practical refers here to the health benefits that may be gained by average person capabilities. Useful information that may be distributed to the system users includes: personal exposure levels, alerts in regard to abnormal air pollution levels, recommendations for means to reduce pollution levels indoors and outdoors and relevant background information. Relevant background information may refer to; information that is personalized to the user need and to the specific pollution condition. By this the system may bring a complete informative solution to users in regard to air pollution. Pollution is a complex and dynamic subject. As in any complex and dynamic areas, proper decision making must be based on situational awareness. Situation awareness (SA) is the perception of environmental elements within a volume of time and space, the comprehension of their meaning, and the projection of their status in the near future. In respect to pollution, SA means the perception of personal impact of pollution levels types and impacts. The expected outcome in regard to pollution is indication of an action that needs to be taken to reduce pollution exposure levels and thus protect personal health. Providing these to people globally is the system mission to support the stated above objective.

In order to achieve these goals, a sensing device for measuring pollution rates is disclosed. The device comprises a sensing unit, comprising at least one sensor, a data acquisition and processing unit, a communication unit, and a power unit. According to some embodiments of the present invention the sensing device may further comprise at least one sensor, selected from a group comprising: air pollution sensors, for sensing gasses and for sensing particles by size; noise sensors; radiation sensors water pollution sensors and atmospheric condition sensors.

According to some embodiments of the sensing device the acquisition and processing unit may comprise an analog to digital converter, to translate the sensors' readings to digital format. The device may further comprise a real time clock for time tagging the sensors readings, a memory unit to store the device's operating software and sensor readings, and a microprocessor.

According to some embodiments of the present invention, the sensing device may comprise an external power source. The external power source may be, according to yet other embodiments of the present invention, an energy scavenging device selected from a group comprising: solar panel, wind electricity generator and vibration-based electricity generator.

Another embodiment of the present invention may comprise a personal health monitoring system comprising: at least one outdoor sensing unit, at least one indoors sensing unit, a communication device, a server, and a database. According to one embodiments of the present invention the at least one outdoor sensing unit, the at least one indoor sensing unit, the server and the database are in active communication with each other via a communication network.

According to some embodiments of the present invention, each of the at least one outdoor sensing unit and the at least one indoor sensing unit may comprise a plurality of sensors selected from a group comprising: air pollution sensors for sensing gasses and for sensing particles by size, noise sensors, radiation sensors, water pollution sensors and atmospheric condition sensors.

According to some embodiments the server of the above system may comprise a processor to process raw data received from the sensing units. According to some embodiments of the present invention, the processor is adapted to calibrate raw data received from the sensing units. According to alternative or additional embodiments of the present invention, the processor is adapted to analyze data received from the sensing units and send the analyzed data to at least one user interface device, wherein the at least one user interface device is selected from a group comprising: a personal computer, a laptop computer, a mobile communication device and a website accessible via a communication network.

The database of the system may comprise a lookup table including sensed pollutants rates and corresponding recommended measures to reduce exposure to the sensed pollutants.

A method according to the present invention, for indoor pollution source identification, is also disclosed, the method comprising: receiving indoor pollution data from an indoor sensing unit. Receiving outdoor pollution data from at least one outdoor sensing unit located in the vicinity of the indoor sensing unit. Comparing the indoor pollution data and the outdoor pollution data based on common time scale and concluding, based on said comparison, weather the pollution source is inside the indoor space, or outside the indoor space. According to some embodiments of the present invention, the location of the source of pollution may also be detected.

According to another embodiment of the present invention, a method for personal accumulated pollution exposure levels estimation is disclosed, the method comprising: creating a time log of the user location information, creating pollution data tables containing location information, time information and pollutant information, integrating said location information, said time information and said pollution information to conclude the accumulated exposure over a predefined period of time, and accumulating pollution exposure levels in terms of level per period of time.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram of a sensing unit according to embodiments of the present invention.

FIG. 2 is a schematic illustration of a personal health monitoring system according to an embodiment of the present invention;

FIG. 3 is high level over all data aggregation and distribution schema of a system according to some embodiments of the present invention.

FIG. 4 is a flow chart of an indoor pollution source identification method according to one embodiment of the present invention;

FIG. 5 is a flow chart of a personal accumulated pollution exposure levels estimation method according to some embodiments of the present invention; and

FIG. 6 is a flow chart of a pollution counter measures recommendation method according to embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

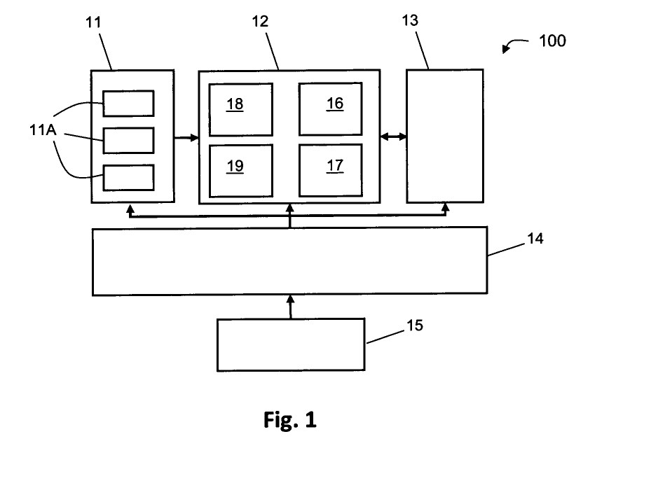

FIG. 1 is a block diagram of a sensing unit 100 according to some embodiments of the present invention. Sensing unit 100 may be either an indoor sensing unit or an outdoor sensing unit. Sensing unit 100 may comprise a sensing section 11 that may include a plurality of pollution sensors 11A for sensing and measuring pollutants rates of a variety of pollution types. Sensors 11A may consist of air pollution sensors, for sensing gasses, such as: nitrogen dioxide (NO₂), nitrogen oxide (NO), carbon oxide (CO), Sulfur dioxide (SO₂), ozone (O₃), and Volatile organic compounds (VOC), and for sensing particles by size, such as: 10 micron, 2.5 micron and nano-metric scale particles. It would be appreciated that other air pollutants may be measured in addition or alternatively.

Sensors 11A may further consist noise sensors, radiation sensors, water pollution sensors and atmospheric condition sensors for sensing ambient temperature, barometric pressure, relative humidity, UV radiation (of preferably all types) and other or additional atmospheric conditions. It would be appreciated by those skilled in the art that other or additional pollution sensors may be used. It would be appreciated by those skilled in the art that when sensing unit 100 is adapted for indoor use sensors 11A may be designed to operate and monitor pollution at indoor environments. The sensing capabilities may target indoor pollutants such as tobacco smoke, formaldehyde, volatile organic compounds, radiation etc. Sensors 11A may be designed to work at common indoor space such as home, office, public indoor space etc. In such configuration the communication of sensing unit 100 a communication network such as the Internet may be based on commonly available Internet accesses points both wire-based (copper or optical or both) and wireless.

It would be further appreciated that when sensing unit 100 is adapted for operation as an outdoor sensing unit, sensors 11A may be designed to work at and monitor the outdoor environment. The sensing capabilities may target common outdoor pollution factors such as outdoor air pollution, noise, radiation etc.

Sensing unit 100 may further comprise a data acquisition and processing unit 12. Acquisition and processing unit 12 may perform the sensors sampling and device general management, however at least some of the sensors include signal sampling and communication capabilities. Acquisition and processing unit 12 may comprise an analog to digital converter 18 to translate analog readings of sensors 11A to digital format, a real time clock 17 for time tagging, for example of signal sampling of sensors 11A, a memory unit 19 to store the device operating software, system and/or user changeable parameters, and sensor readings, microprocessor 16 for managing the device and other components needed for computing functionality, as known in the art. As illustrated in FIG. 1, sensing unit 100 may further comprise a communication unit 13. Communication unit 13 may be adapted to communicate with external devices and networks, such as USB channels, telephone channels (land line and/or cellular) and network channels. Communication unit 13 may further provide connectivity of sensing unit 100 to a communication network such as the Internet, and any other software and hardware known in the art which is required in order to allow communication between sensing unit 100 and a communication network.

Sensing unit 100 may further comprise a power unit 14 delivering electrical power to each of the other units of sensing unit 100. Power unit 14 may comprise an energy storage component 14A such as a battery or a capacitor. Energy storage component may be rechargeable. Power unit may be adapted to provide the required power to the units of system 100.

According to some embodiments of the present invention, power unit 14 may be further adapted to be powered by external power sources 15, such as grid power or energy scavenging device that may be adapted to charge the energy storage component 14A. According to some embodiments of the present invention, external power source 15 may be a solar panel, wind or vibration electricity generation device etc.

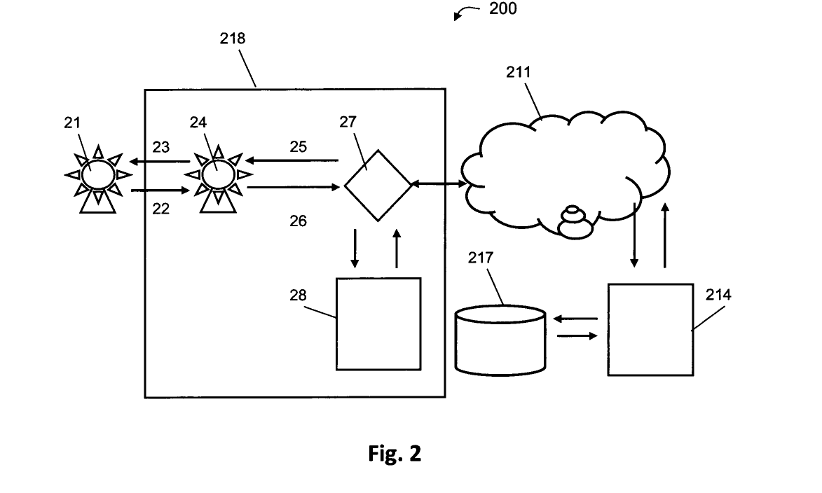

Reference is now made to FIG. 2 which is a schematic illustration of a personal/local health monitoring system 200 according to an embodiment of the present invention. Monitoring system 200 may comprise at least one outdoor sensing unit 21, at least one indoors sensing unit 24, a communication network, such as the Internet 211, and interfacing means thereto, such as Internet hub 27.

Monitoring system 200 may further comprise server 214 and database 217 stored on storage device such as memory unit 19 (FIG. 1) to allow data accumulation from at least one outdoors sensing unit 21 and at least one indoors sensing unit 24 and to store the data, analyze the data and circulate information to other end users.

As may be seen in FIG. 2, at least one outdoor sensing unit 21, such as sensing unit 10 described in FIG. 1 above, may operate at the geographical close vicinity of indoor space 218. Outdoor sensing unit 21 may be in active communication with indoor sensing unit 24 and/or in active communication with server 214 in bidirectional communication channels 22, 23. Outdoor sensing unit 21 may receive information originating from server 214, such as time synchronization messages and software updates or other special inquiries from server 214. Sensing unit 21 may send data such as sensor unit ID, sensor unit health status, pollution readings (e.g. levels of Ozone, Nitrogen Dioxide, noise, RF radiation and the like), real time/reading time clock, location and meteorological conditions (e.g. temperature, humidity and the like). The data from outdoor sensing unit 21 may be sent directly to server 214 via hub 27 and communication network 211 or through a mediator device that may be, according to some embodiments, indoor sensing unit 24. According to some embodiments of the present invention, mediator device, such as indoor sensing unit 24, may be connected to a dedicated or non-dedicated communication device 28, which in turn may be connected to communication network 211 via hub 27. Communication device 28 may be a Personal Computer (PC), a Personal Digital Assistant (PDA), a telephone, a mobile phone such as a smart phone, or any other communication device capable of communicating with another communication device via a communication network such as the internet.

Indoor sensing unit 24, placed in indoor space 218, may also communicate bidirectionally (25, 26) with server 214 similarly to outdoor sensing unit 21 (receiving time synchronization messages and sending sensor readings and other information to server 214). In addition, indoor sensing unit 24 may serve as communication mediator to outdoor sensing unit 21 as explained above. It would be appreciated by those skilled in the art that the communication between all or some of the above elements (i.e. outdoor sensing unit 21, indoor sensing unit 24, server 214) may be wire communication and/or wireless communication.

Hub 27 may be a conventional gateway to communication network 211. It can be either through cable connection and a modem or wireless internet hub or any via other communication channel known in the art. The data from and to sensing units 21 and 24 may be routed through hub 27.

Communication device 28 may serve as a user interface (UI) to the system, presenting the retrieved and processed data from the sensors and enabling operation of the system features. The user interface may be dedicated software installed on communication device 28 or may be a website accessible from communication device 28.

According to some embodiments of the present invention, communication device 28 may take part in the communication routing between indoor sensing unit 24 and server 214 and/or between outdoor sensing unit 21 and server 214. Sensing units 21 and 24 may be connected to communication device 28 via any connection means know in the art, such as Universal Serial Bus (USB) connection. It would be appreciated that other means of connection may be used.

As may be seen in FIG. 2, server 214 may receive data from sensing units 21 and/or 24 and may send data massages to sensing units 21 and 24. The data sent to sensing units 21 and 24 may include time synchronization messages, software updates, instructions and other software-based functionally targeting elements (e.g. sensor sampling scheduling).

According to some embodiments of the present invention server 214 may receive raw data from sensing units 21 and 24, atmospheric conditions data and pollution data from sources that are not received via the system's sensing units, such as municipal monitoring units, university research units, etc. Server 214 may use calibration tables and mathematical functions (group theorem etc.) to modify raw data received from the sensors to receive more accurate data or data modified for purpose such as trending, long-term data cumulating and others. For example, accuracy of readings from some sensors may depend on the relative humidity and ambient temperature at the vicinity of those sensors (e.g. metal oxide gas sensors).

Server 214 may adjust the sensor readings based on the sensor ID (that may contain the sensor's part number or any other unique ID data) and calibration data that may be provided by the sensor manufacturer. Performing this feature at server 214 (and not on the sensor itself) is possible because of the overall system design; data from the sensors is retrieved to the users after it has been processed at server 214. The importance of this feature is in reducing the complexity of the sensing units 21 and 24, hardware and software, therefore reducing their cost of manufacturing. The last is important to support large scale deployment of the sensing units 21 and 24.

According to some embodiments of the present invention, when a new sensing unit 21 or 24 is added to the system and new entry to the database is created the server software may perform a search to learn what other sensors exist in the geographical vicinity of new sensing unit 21 or 24, based on range definitions. Server 214 may use all available geospatial information both retrieved from neighboring sensing units 21 and 24 and from information in the public domain, such as municipal air monitoring stations, pollution sources, pollution next to roads and transportation lines. According to one embodiment of the present invention, server 214 may register all sensing units at distance X (i.e. neighboring sensing units), thus creating a cluster of sensors. Server 214 may further register nearby air quality monitoring stations that are not part of the system's sensing units. These stations may include, beside pollution sensors, also meteorological sensors like wind speed and direction. For example in the USA it would be the Environmental Protection Agency (EPA) network called “Airnow”.

Server 214 may further register pollution sources inventory like: power plants, chemical production plants or refineries and nearby transportation routs such as highways, ports and the like. Server 214 may further register the distance of these known pollution sources from the new sensing unit. In many countries and jurisdictions this information is in the public domain.

It would be appreciated that the information received from neighboring sensing units, from other quality monitoring stations and from the public domain may provide information for adjusting the data received from a new sensing unit and for calibrating the new sensing unit.

According to some embodiments of the present invention, server 214 may be in active communication with a storage unit storing database 217. The data received from sensing units 21 and 24 may be stored in database 217. According to some embodiment of the present invention, database 217 may include data and information received from other sources. According to some embodiments of the present invention, database 217 may save a lookup table including sensed pollutants rates and recommended reaction to reduce hazard.

Computations performed on server 214 may extract pollution information from the sensed data and search for the recommended counter measure to the pollution as reflected in the extracted information and may send recommendation of these counter measures to end users.

According to yet another embodiment of the present invention, system 200 may provide vehicle users climate control information in regard to the ambient pollution levels outside the vehicle. According to this embodiment indoors space 218 may be a vehicle's cabin having a climate control system 28 that may control the fresh/outside air intake to vehicle cabin 218 based on the pollution levels received from sensing units 21 and 24. If the pollution levels outside vehicle cabin 218 are lower than a definable threshold fresh air may be intaked and vice versa.

According to some embodiments of the present invention the climate control information may be received at the climate control via wireless communication channel (cellular, Wi-Fi, Wi-Max etc.). The data may be sent at near to real time manner from sensing units 21 and 24 to server 214 and after analysis, the pollution information may be sent to vehicle 218, as much as to any other user/client. Location data of the vehicle can be sourced either from the cellular operator or from onboard vehicle navigation instruments such as GPS (global positioning system) (not shown). The system can include in the vehicle's cabin air quality sensing unit 24 for more robust control over the intake fresh air management. However it would be appreciated by those skilled in the art that sensing unit 24 may not be required and information regarding outdoor pollution in vehicles 218 vicinity may be derived from outdoor sensing units 21 in the vicinity of vehicle 218, from local atmospheric information (wind direction and speed, inversion level, etc.) and information regarding the location of vehicle 218.

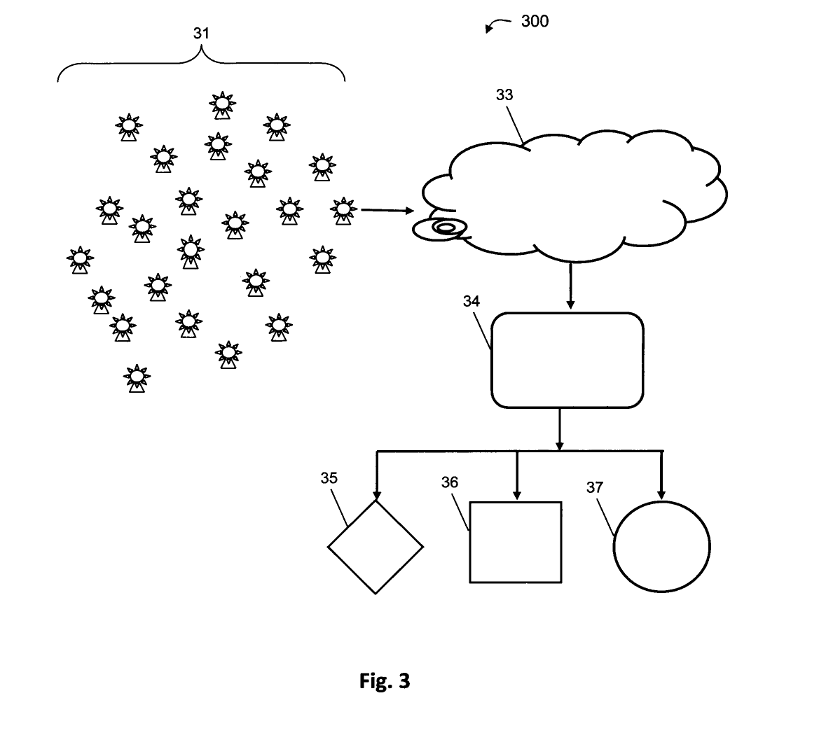

FIG. 3 is high level overall data aggregation and distribution scheme of system 300 for personal health monitoring based on multitude-points ambient quality data according to some embodiments of the present invention. As may be seen in FIG. 3, a plurality of pollution sensing units 31 may be located in a plurality of geographical locations. The geographical locations may be indoor and/or outdoor locations. Plurality of sensing units 31 may send pollution data 32, over a communication network 33 such as the Internet, to a centralized server 34. Data sent from sensing units 31 may be based on sampling periods and may include: unit ID, unit health status, pollution sensors readings, real time clock value, location of the sensing unit and meteorological conditions (e.g. temperature, humidity and the like) in the vicinity of the sensing unit. Sensing units 31 may transmit the data to the server via one or more communication networks 33, such as the Internet. The data may reach communication network 33 through indoor Internet hub (not shown) either wirelessly or by wire. Sensing units 31 may be installed by users at their living places (homes, offices kindergarten schools, vehicles etc.) and therefore are designed to connect to the Internet by commonly available Internet connection devices. Sensing units 31 may be adapted to allow automatic initialization and connection of each sensing unit to network 33 (i.e. plug and play capabilities) thus allowing each sensing unit 31 to serve as an independent entity capable of communicating with other sensing units and with server 34 to send and receive data therebetween. Furthermore, the connection of each sensing unit 31 to communication network 33 may allow each sensing unit 31 to be accessed via network 33.

It would be appreciated by those skilled in the art that each sensing unit plug and play capabilities may allow easy installation by an end users and may facilitate wide deployment of sensing units 31.

Location of each sensing unit 31 may be determined by one or more of the following methods: GPS receiver; wireless communication triangulation methods (cellular or Wi-Fi); manually by the user or at the assembly line based on the user delivery address. The last enable cost reduction of the sensing devices and support large scale deployment. According to one embodiment of the present invention, sensing unit 31 may have a Wi-Fi module that is preset to peer to peer mode, to allow it to communicate directly with a Smart phone or any other wirelessly connectable device, via the Wi-Fi channel. Once the peer to peer connection with the Smart phone is established, the Smart phone may transmit data to sensing unit 31, such as the security information needed to connect to the home wireless router and get out to the Internet (SSID) and the location data of the Smart phone.

Server 34 comprises a processor (not shown) and storage means (not shown) for processing and storing and retrieving data received from sensing units 31. The processed data may then be distributed to end-users 35, 36 and 37. The processed data may be distributed to the system in the form of various services via the Internet and cellular networks.

The processed data may be sent to user's mobile devices, personal computers, PDA's, laptops, tablet computers, mobile phones and the like and/or to business servers. For each end user specialized information formats/message may be used because of the differentiation both in need and communication method. For instance when the processed data is sent to an end user's cellular devices the information may be sent over the cellular network or other communication methods to mobile devices (e.g. Wi-Fi, Wi-Max etc.). The information sent may include the information needed to support the system's mobile device features. Alternatively or additionally, information may be sent over the internet and presented on the system webpage to support the system Internet features. The information may be viewed and used by any conventional browsing technique both from stationary devices and mobile devices. The information may be further sent to other businesses clients over the internet for example pollution mapping facilities sent to weather news Company. Other interface options may include installing costume-made software at the user end device to receive the system information, such as via a specialized application to a smart phone.

According to some embodiments of the present invention, system 300 enables users to personally configure data retrieved from sensing units 31 based on user special consideration such as medical condition (i.e. asthma, cardiovascular diseases), baby at home, active in sport etc. The personal configuration may result in a change in the thresholds for warnings and in specifically adjusted recommendation relevant to the various indoor and outdoor pollutants. Thus the information the user receives from sensing units 31 may be more precise and relevant to that user. Personal configuration may be done manually by the user in the system user interface (not shown) without involving the sensing unit 31 itself. This feature enables simplified and low cost sensing unit 31 design and the ability to give personalized data from various non unitized sensing units.

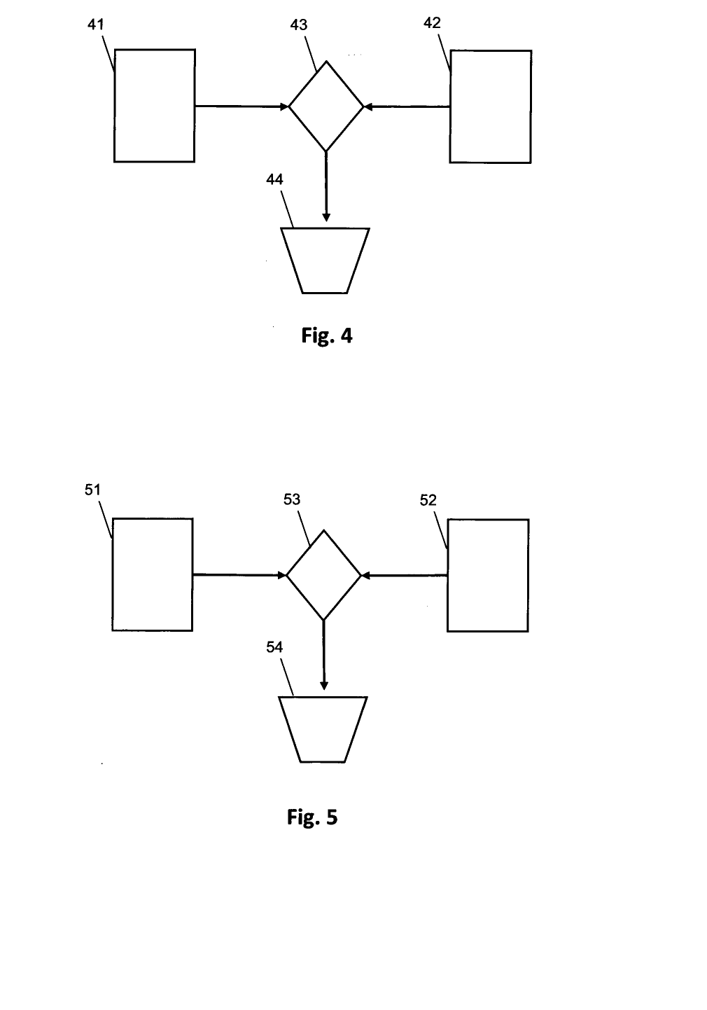

FIG. 4 is a flow chart of an indoor pollution source identification method according to an embodiment of the present invention. The method comprises the following steps: receiving indoor pollution data [block 41] and outdoor pollution data [block 42] categorized by the various pollutants and their time/date tags. The received outdoor sensor pollution readings may be received from a sensor or sensors at the geographical vicinity of the indoor sensor from which the indoor pollution data is received.

Comparing of indoor and outdoor sensor readings based on common time scale to conclude whether the pollution originates from inside the indoor space, or originates from the outside [step 43] and determining a pollutant or a plurality of pollutants source(s) [step 44].

The plurality of sensors in the system both from the user itself and form multiple users, support the capability of performing automated data comparing between the various sensors may lead to automated identification of the pollution source indoor and outdoor. For example if the indoor pollution data for a specified gaseous pollutant is significantly higher than the outdoor pollution data received for the same pollutant over a predefined period of time then it may be deduced, with high degree of certainty, that the source is an indoors source of pollution. Additional mathematical functions for sources identification may be used, such as functions which are based on experimental data and comparison to past readings and other stored information. For example, if indoor humidity levels are continually higher than outdoor levels then the source of the measured humidity is most likely located indoor and could be due to either a leaking tap/piping or condensation over poorly insulated window (it should be known that humidity control is critical to prevent mold, which is an important indoor air quality factor). Examination of the temperature difference between indoor and outdoor or user interaction could specify the source once more.

Furthermore the method enables identification of indoor gaseous. For example, if outdoor SO₂ levels sensed by neighboring outdoor sensing units are significantly lower than indoor sensor readings source might be kerosene or coal heater. According to some embodiments of the present invention, further determination of the precise source is done with user interaction based on pre prepared questionnaire for the user sent by the system via the user interface.

In case of indoor pollutant assessment the output of the process may include indication of indoor source options that can be taken by the user in order to minimize the negative effect of the pollutant. In case of outdoor assessment the output may include indication of optional pollution sources based on gradients analysis of pollution data at the area where the indoor location is. This will enable estimation of the outdoor pollutant source, such as a nearby highway or electricity generation plant.

FIG. 5 is a flow chart of a method for estimation of personal accumulated pollution exposure levels according to some embodiments of the present invention, the method comprising the following steps:

Creating a time log of the user location history [block 51]. The location history time log may be organized in a table containing location data at specified time resolution (i.e. location every 1 second for example). According to some embodiments of the present invention the location information can be extracted from the cellular operator based on location services capabilities or from user devices such as GPS or other navigation devices. According to some embodiments of the present invention the sensors readings may be aggregated and logged at a database with the optional slicing by: time, location (e.g. UTM coordinates or the like) and magnitude of various pollutants levels.

Pollution data tables containing information sliceable according to, for example, location, time and pollutant [block 52] may be created based on geospatial time tagged pollution data and personal location data. The information for creating the pollution data table may be extracted from the plurality of sensors that are part of the system according to embodiments of the present invention.

Integrating the location information and the pollution information to conclude the accumulated exposure over a predefined period of time [block 53]. In order to calculate personal accumulated exposure levels, the location information (i.e. coordinate and time) may be integrated with the pollution data in the database to generate exposure data; time and pollution levels meaning accumulated exposure. According to some embodiments of the present invention the location data can include speed and acceleration data as a first and second derivative of the location data.

Accumulating pollution exposure levels in terms of level per period of time (week, month, year, etc.) [block 54]. The importance of accumulated exposure levels is due to the standards that are in unit per month. It would be appreciated that the exposure period multiplied by the measured pollutant level equals to accumulated exposure levels.

The method may further provide a warning to a user, via user interface, indicating that he or she are exposed to high levels of pollution or are in a polluted area or are entering a polluted area, based on user location information and real time or near to real time geospatial pollution data. The user location information may be based on mobile location services provided by the cellular operator and may be integrated with the geospatial pollution information to produce the worming when needed. It would be appreciated by those skilled in the art that other localization methods may be used.

In yet another method according to embodiments of the present invention a warning may be produced both in “push” method or based on user inquiry. ‘Push’ stands for warning sent to the user automatically by the system when the user's preset definitions are met, the system may analyze the location and pollution data continuously and may send warnings based on the thresholds determined by the system in the light of the user system personalization. User inquiry may refer to user performing inquiry at a specific time and/or location. the Location could be the user's own location or a location the user is not present in such as: kindergarten, school, home, office etc. The system may analyze continuously the pollution levels in the specified location and send warnings based on the user's preset definitions. In yet another embodiment of the present invention, the user may receive a warning indicating the current pollution levels at the current user location at the time of inquiry. The warning format may be user definable and may include, among other formats: text massages, voice massages, video massages, emails, animations etc.

According to other embodiments of the present invention, route planning information may be sent to the user in order to reduce accumulated exposure levels, based on an optimization algorithm that receives geospatial pollution data and mapping and traffic data commonly used in navigation applications, and returns a route between a first user defined location A and a second user defined location B with minimum pollution exposure. Minimum pollution exposure is the minimum value of the integration along time and expected pollution levels. The pollution levels are predicted by the system based on prolonged data analysis in the specified geographical area, based on pollution pattern behavior (prediction). The route may be performed by any sort of motion: by vehicle, by foot, jogging etc. The initiation of the route planning is either user determined (per user request) or initiated by the system due to high accumulated exposure levels.

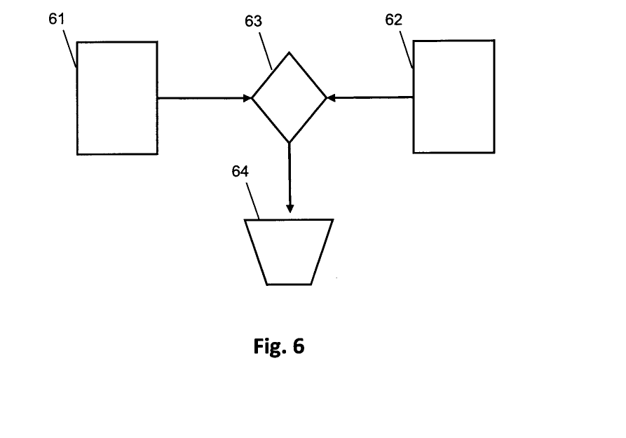

Reference is now made to FIG. 6 which is a flow chart of a pollution counter measures recommendation method according to embodiments of the present invention, the method comprising:

Gathering pollution sensor readings of specified geographical location associated with specific system user [block 61], the readings may be gathered form either indoor sensing units and/or outdoor sensing units. The gathered data is for a predefined period of time sufficient in order to represent the pollution conditions in the specified location. The data may be sliced with time pollution type and level.

Providing a database of pollution counter measures organized in tables sliced by pollution type, pollution allowed levels, as reflected from standards and information received from users, and possible measures to reduce the pollution [block 62]. The database may store additional data to support the suitability of a counter measure to the user, such as: the expected cost of applying the counter-measure, technology, power consumption, sustainability etc.

Comparing the data gathered by indoor and outdoor sensing units with the data stored in the database in order to extract a recommendation on counter measures to the pollution levels in the users' location [block 63]. The recommended measure may be the counter measure that received the highest rank based on the pollution sensor readings and the user preset instructions (for example cost range of solution or anti-pollution technology available to user). The recommendations are based on the prolong sensors readings that are processed automatically for recommending the type of measure needed to be taken in order to reduce the pollution levels. For example if ozone levels are high (compared to the recommended levels) the system would recommend to place an active carbon filtration system indoor. According to some embodiments of the present invention, the data provided from the system will include specific instruction in regard to functionality (location, sizes, operation modes and timings etc. . . . ), business related information such as: were to buy, links to suppliers, scientific background information and other relevant information such as information that addresses the pollution situation and the personal condition of the user. For example if the user suffers form asthma and the indoor space he is in (home, office etc.) has high Ozone levels the user will receive information such as medical studies, articles and recommendations on treatments scientific/medical discoveries and other news material that can promote the user understanding and cooping in his specific situation. According to some embodiments of the present invention the information may be distributed to the user in one or more methods such as emails, banners on a webpage, portal tabs or any other method known in the art.

When receiving data indicative of high values of ozone indoors, different counter measures may be appropriate such as placing an active carbon filtration system indoor. If, however, a user is interested in low-cost solutions only, the output recommendation may be the type and size of plants needed in order to reduce the pollution.

According to yet another method of the present invention the recommendation may include ventilation instruction. According to this method, indoor air quality data and outdoor air quality data and economical consideration factors such as electricity costs are received and instructions regarding indoor space optimized ventilation are sent to the user. Optimization of the ventilation instruction is in timings over the day, duration of the ventilation, control of opening/closing air-openings like windows. The objective of the optimization is to reduce to the minimum the polluted air that enters the indoor environment and to reduce to minimum the energy associated with ventilation. According to some embodiments of the present invention, the ventilation instructions are either to the user to perform manually or done automatically as part of smart home apparatus.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A sensing device for measuring pollution comprising:
a sensing unit comprising at least one sensor;
a data acquisition and processing unit;
a communication unit; and
a power unit;

2. The sensing device according to claim 1 wherein said at least one sensor is selected from a group comprising: air pollution sensors, for sensing gasses and for sensing particles by size; noise sensors; radiation sensors water pollution sensors and atmospheric condition sensors.

3. The sensing device according to claim 1 wherein said acquisition and processing unit comprises:
an analog to digital converter, to translate sensors readings to digital format;
a real time clock for time tagging to sensors readings;
a memory to store the device operating software and sensor readings; and
a microprocessor.

4. The sensing device according to claim 1 wherein said power unit comprises:
an energy storage component; and
a voltage differentiation device;
wherein said voltage differentiation device is adapted to provide different voltage to different components of said sensing apparatus.

5. The sensing device according to claim 4 wherein said power unit further comprises a connector to enable connection to an external power source.

6. The sensing device according to claim 5 wherein said external power source is an energy scavenging device selected from a group comprising: solar panel, wind electricity generator and vibration electricity generator.

7. A personal health monitoring system comprising:
at least one outdoor sensing unit;
at least one indoors sensing unit;
a communication device;
a server; and
a database;
wherein said at least one outdoor sensing unit, said at least one indoor sensing unit, said server and said database are in active communication with each other via a communication network.

8. The system according to claim 7 wherein each of said at least one outdoor sensing unit and said at least one indoor sensing unit comprises a plurality of sensors selected from a group comprising: air pollution sensors, for sensing gasses and for sensing particles by size; noise sensors; radiation sensors water pollution sensors and atmospheric condition sensors.

9. The system according to claim 7 wherein said server comprises a processor to process raw data received from said sensing units.

10. The system according to claim 9 wherein said processor is adapted to calibrate raw data received for said sensing units.

11. The system according to claim 9 wherein said processor is adapted to analyze data received for said sensing units and send said analyzed data to at least one user interface device.

12. The system according to claim 11 wherein said at least one user interface device is selected from a group comprising: a personal computer, a laptop computer, a mobile communication device and a website accessible via a communication network.

13. The system according to claim 7 wherein said database comprises data received from said sensing units and data received from other sources.

14. The system according to claim 7 wherein said database comprises a lookup table including sensed pollutants rates and recommended measures to reduce exposure to said sensed pollutants.

15-21. (canceled)

21. A method for indoor pollution source identification, the method comprising:
receiving indoor pollution data form an indoor sensing unit;
receiving outdoor pollution data from at least one outdoor sensing unit located in the vicinity of said indoor sensing unit;
comparing said indoor pollution data and said outdoor pollution data based on common time scale;
concluding, based on said comparison weather the pollution source is inside the indoor space, or outside said indoor space.

22. The method according to claim 21 wherein when the pollution source is indoors, indoor pollution source options are sent to a user.

23. The method of claim 22 further comprising the step of sending a user a questionnaire for determination the precise source of pollution.

24. The method of claim 21 wherein when the source of pollution is outdoors, sending a user optional pollution sources based on gradients analysis of pollution data at the area where the indoor location is.

25-38. (canceled)
